(19) United States
(12) Patent Application Publication
(10) Pub. No.: US 2025/0356989 A1
(43) Pub. Date: Nov. 20, 2025

(54) VIRTUAL TESTING OF HARDWARE AND SOFTWARE FEATURES FOR MEDICAL IMAGE ACQUISITION DEVICES

(71) Applicant: Siemens Healthineers AG, Forchheim (DE)

(72) Inventors: David Grodzki, Erlangen (DE); Dorin Comaniciu, Princeton, NJ (US); Boris Mailhe, Plainsboro, NJ (US); Mariappan S. Nadar, Plainsboro, NJ (US); Birgi Tamersoy, Erlangen (DE); Peter Gall, Uttenreuth (DE); Jens Gühring, Erlangen (DE); Steffen Schröter, Fürth (DE); Rainer Schneider, Höchstadt (DE); Thorsten Speckner, Erlangen (DE)

(21) Appl. No.: 18/664,521

(22) Filed: May 15, 2024

(51) Int. Cl. G16H 30/20; G06T 7/00

(52) U.S. Cl. CPC G16H 30/20; G06T 7/0014

(57) ABSTRACT

Systems and methods for determining a target imaging protocol for an image acquisition are provided. At least one of 1) one or more performance indicators, 2) an input imaging protocol for an image acquisition, or 3) changes in conditions of the image acquisition are received. A target imaging protocol is determined using an image acquisition model based on the at least one of the one or more performance indicators, the input imaging protocol, or the changes in the conditions of the image acquisition. The target imaging protocol are output.

### TECHNICAL FIELD

The present invention relates generally to testing of features for medical image acquisition devices, and in particular to virtual testing of hardware and software features for medical image acquisition devices.

### BACKGROUND

Medical images are acquired using medical image acquisition devices for diagnosing and treating medical conditions in patients. Medical image acquisition devices have improved over time, bringing about new software and/or hardware features. Before being released, such new software and/or hardware features are typically tested to identify potential bugs and to verify that images are generated as intended without introducing artefacts. For example, before a new MRI (magnetic resonance imaging) technique is released, this new MRI technique is tested and adapted for all MRI scanner types and targeted software versions. However, conventional testing of features of medical image acquisition devices is time consuming and costly.

### BRIEF SUMMARY OF THE INVENTION

In accordance with one or more embodiments, systems and methods for determining a target imaging protocol for an image acquisition are provided. At least one of 1) one or more performance indicators, 2) an input imaging protocol for an image acquisition, or 3) changes in conditions of the image acquisition are received. A target imaging protocol is determined using an image acquisition model based on the at least one of the one or more performance indicators, the input imaging protocol, or the changes in the conditions of the image acquisition. The target imaging protocol are output.

In one embodiment, the input imaging protocol is received. The input imaging protocol is for a reference medical image acquisition device. An image is generated using a computation image acquisition model based on the input imaging protocol. The generated image is compared with a medical image generated by the reference medical image acquisition device. The input imaging protocol is iteratively adjusted based on the comparison to determine the target imaging protocol. The target imaging protocol is for a target medical image acquisition device. In one embodiment, the input imaging protocol is iteratively adjusted by backpropagation. In another embodiment, the input imaging protocol is iteratively adjusted by gradient descent on an output of a critic network. The critic network is trained to predict the one or more performance indicators. In another embodiment, the input imaging protocol is iteratively adjusted by an actor network to optimize a critic loss. The critic loss is calculated based on 1) the one or more performance indicators determined based on the comparison and 2) the one or more performance indicators predicted by a critic network.

In one embodiment, the input imaging protocol and the one or more performance indicators are received. The input imaging protocol is for a reference medical image acquisition device. The target imaging protocol is determined using a machine learning based image acquisition model. The machine learning based image acquisition model receives as input the input imaging protocol and the one or more performance indicators and generating as output the target imaging protocol. The target imaging protocol is for a target medical image acquisition device.

In one embodiment, the input imaging protocol and the changes in the conditions of the image acquisition are received. The input imaging protocol is for an initial condition of the image acquisition. The target imaging protocol is determined using a computational image acquisition model based on the input imaging protocol and the changes in the conditions of the image acquisition. The target imaging protocol is for a target condition of the image acquisition.

In one embodiment, the input imaging protocol and the changes in the conditions of the image acquisition are received. Events are determined using a computational image acquisition model based on the input imaging protocol and the changes in the conditions of the image acquisition. The target imaging protocol may be determined based on the events.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining a target imaging protocol from an input imaging protocol modeling using an image acquisition model modeling new features of a medical image acquisition device, in accordance with one or more embodiments;

FIG. 2 shows a workflow for determining a target imaging protocol for a target medical image acquisition device from an input imaging protocol for a reference medical image acquisition device using a computational image acquisition model of the target medical image acquisition device, in accordance with one or more embodiments;

FIG. 3 shows a workflow for optimizing an imaging protocol by differentiable simulation to determine a target imaging protocol, in accordance with one or more embodiments;

FIG. 4 shows a workflow for optimizing an imaging protocol by differentiable critic to determine a target imaging protocol, in accordance with one or more embodiments;

FIG. 5 shows a workflow for optimizing an imaging protocol by reinforcement learning to determine a target imaging protocol, in accordance with one or more embodiments;

FIG. 6 shows a workflow for determining a target imaging protocol for a target medical image acquisition device from an input imaging protocol for a reference medical image acquisition device using a machine learning based image acquisition model of the target medical image acquisition device, in accordance with one or more embodiments;

FIG. 7 shows a workflow for determining a target imaging protocol for a medical image acquisition device for target conditions of the image acquisition from an input imaging protocol for the medical image acquisition device using a computational image acquisition model of the medical image acquisition device, in accordance with one or more embodiments;

FIG. 8 shows a workflow for determining real time events for a medial image acquisition device according to an input imaging protocol and image acquisition conditions, in accordance with one embodiment;

FIG. 9 shows a workflow for training and applying an AI agent for predicting a target imaging protocol, in accordance with one or more embodiments;

FIG. 10 shows an exemplary artificial neural network that may be used to implement one or more embodiments;

FIG. 11 shows a convolutional neural network that may be used to implement one or more embodiments;

FIG. 12 shows a data flow diagram according to an embodiment for using a generative adversarial network to implement one or more embodiments;

FIG. 13 shows a schematic structure of a recurrent machine learning model that may be used to implement one or more embodiments; and

FIG. 14 shows a high-level block diagram of a computer that may be used to implement one or more embodiments.

### DETAILED DESCRIPTION

The present invention generally relates to methods and systems for virtual testing of hardware and/or software features for medical image acquisition devices. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for an image acquisition model for modeling new hardware and/or software features a medical image acquisition device and/or a patient. The image acquisition model simulates system behavior and image generation to identify bugs or unexpected behavior for virtual testing of the new hardware and/or software features. The image acquisition model may be implemented as a machine learning based image acquisition model (e.g., a neural network) and/or a computational image acquisition model (e.g., based on a finite-element discretization of the Bloch equations in time and space). Based on simulation results output by the image acquisition model, an input imaging protocol is iteratively adjusted to determine a target imaging protocol that is best suited for the medical image acquisition device with the new hardware and/or software features. Every simulation output generates an image, from which performance indicators (e.g., Signal-to-Noise Ratio, Contrast-to-Noise Ratio of a known structure in the image). The acquisition parameters giving the best performance indicators are selected as the target imaging protocol. Advantageously, virtual testing of new hardware and/or software features of a medical image acquisition device is performed in accordance with embodiments described herein with significantly reduced time and cost as compared with conventional approaches.

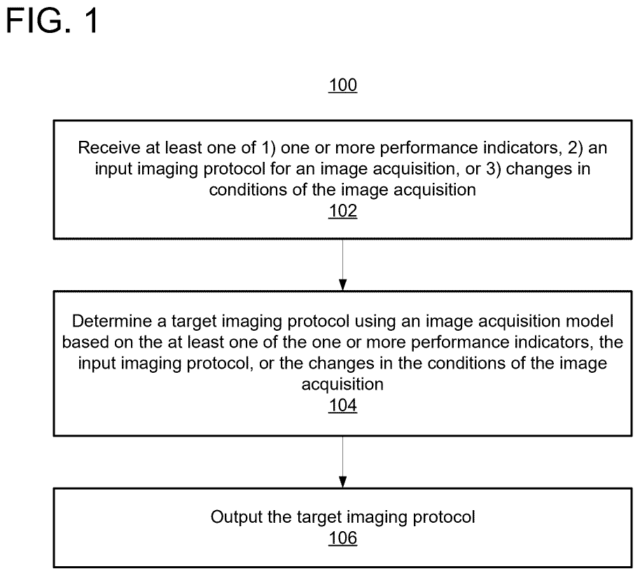

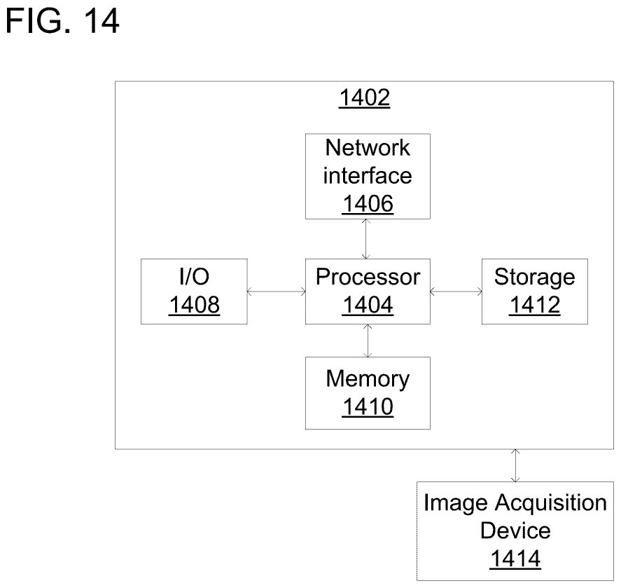

FIG. 1 shows a method 100 for determining a target imaging protocol from an input imaging protocol modeling using an image acquisition model modeling new features of a medical image acquisition device, in accordance with one or more embodiments. The steps and sub-steps of method 100 may be performed by one or more suitable computing devices, such as, e.g., computer 1402 of FIG. 14.

At step 102 of FIG. 1, at least one of 1) one or more performance indicators, 2) an input imaging protocol for an image acquisition, or 3) changes in conditions of the image acquisition are received.

The one or more performance indicators define criteria for determining the target imaging protocol from the input imaging protocol. In one embodiment, the one or more performance indicators define information content criteria, such as, e.g., contrast, SNR (signal to noise ratio), resolution, robustness, etc. In one embodiment, the one or more performance indicators define safety/comfort criteria, such as, e.g., SAR (specific absorption rate), PNS (peripheral nerve stimulation), noise, etc. In one embodiment, the one or more performance indicators define efficiency criteria, such as, e.g., scan time, power, etc. The one or more performance indicators may comprise any other suitable criteria. In one or more, the one or more performance indicators may be weighted based on importance.

The input imaging protocol for the image acquisition defines procedures and parameters for acquiring medical images from a patient using a particular medical image acquisition device. For example, the input imaging protocol may comprise one of more of: patient information of the patient, imaging modality selection, imaging parameters, scan plan, contrast administration, and/or any other suitable parameter for acquiring the medical images. The patient information may comprise patient demographics, relevant medical history, etc. The imaging modality selection may comprise the imaging modality (e.g., CT (computed tomography), MRI (magnetic resonance imaging), US (ultrasound), x-ray, etc.), the anatomical region of interest to be imaged, etc. The imaging parameters define the parameters for image acquisition. For example, for x-ray and CT, the imaging parameters may include tube voltage, tube current, exposure time, slice thickness, reconstruction algorithm, and contrast administration protocols. In another example, for MRI, the imaging parameters may include pulse sequences (e.g., T1-weighted, T2-weighted, diffusion-weighted), repetition time, echo time, field of view, matrix size, slice thickness, and contrast administration protocols. In a further example, for US, the imaging parameters may comprise transducer frequency, gain settings, depth of penetration, focus depth, and doppler settings. The scan plan describes the anatomical region to be imaged, imaging planes (e.g., axial, sagittal, coronal) to be imaged, positioning instructions for the patient, etc. The contrast administration may define the type, dosage, injection rate, and timing of contrast administration. The input imaging protocol may be manually defined by a user (e.g., radiologist) and/or may be automatically defined (e.g., using an artificial intelligence/machine learning based approach).

The changes in the conditions of the image acquisition define changes between the condition of the medical image acquisition device and/or the patient for the input imaging protocol and the condition of the medical image acquisition device and/or the patient for the target imaging protocol to be determined. The changes in the conditions of the medical image acquisition device may include, e.g., changes in field strength (which impacts image contrast, noise level, and RF (radiofrequency) energy deposition), gradient, and RF specifications, which may prevent the input imaging protocol from being played on the target device. The changes in the condition of the patient which may impact imaging may include, e.g., motion/breath hold capability and the presence of metal implants in the body which can perturb the magnetic field.

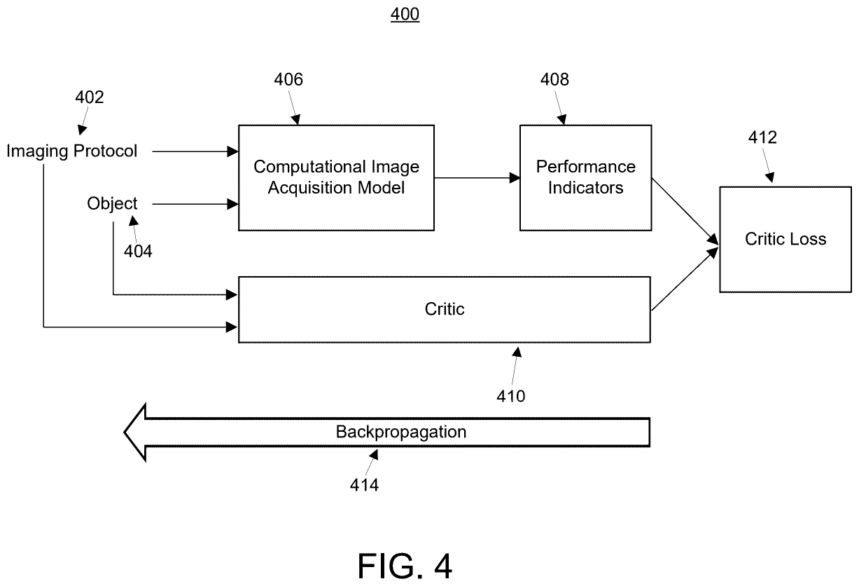

At step 104 of FIG. 4, a target imaging protocol is determined using an image acquisition model based on the at least one of the one or more performance indicators, the input imaging protocol, or the changes in the conditions of the image acquisition.

The image acquisition model may model new hardware and/or software features of the medical image acquisition device, such as, e.g., eddy current behavior, off-center gradient effects, B0 and B1 effects capabilities, and any other relevant aspect of the medical image acquisition device that may have affect the new hardware and/or software features. The image acquisition model may comprise any suitable model of the medical image acquisition device and/or the patient. In one embodiment, the image acquisition model is a machine learning based image acquisition model (e.g., a neural network) trained on a large corpus of training data reflecting a large number of different clinical scenarios. The machine learning based image acquisition model may be implemented according to any suitable machine learning based architecture. In another embodiment, the model is a computational image acquisition model that represents physical relationships between different variables and parameters of the medical image acquisition device and of the patient. The computational image acquisition model may be defined by a set of equations or algorithms. For example, the computational image acquisition model may be defined based on a finite-element discretization of the Bloch equations in time and space.

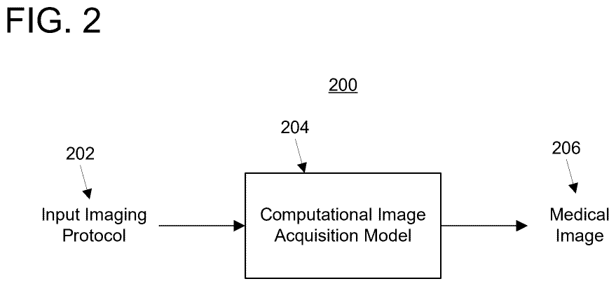

The target imaging protocol may be determined as described below with respect to FIGS. 2 and 6-9.

At step 106 of FIG. 1, the target imaging protocol is output. For example, the target imaging protocol can be output by displaying the target imaging protocol on a display device of a computer system (e.g., I/O 1408 of computer 1402 of FIG. 14), storing the target imaging protocol on a memory or storage of a computer system (e.g., memory 1410 or storage 1412 of computer 1402 of FIG. 14), or by transmitting the target imaging protocol to a remote computer system (e.g., computer 1402 of FIG. 14).

FIG. 2 shows a workflow 200 for determining a target imaging protocol for a target medical image acquisition device from an input imaging protocol for a reference medical image acquisition device using a computational image acquisition model of the target medical image acquisition device, in accordance with one or more embodiments. Workflow 200 may be performed according to method 100 of FIG. 1.

In workflow 200, an input imaging protocol 202 is received. Input imaging protocol 202 is for a reference medical image acquisition device. Input imaging protocol 202 may be received at step 102 of FIG. 1.

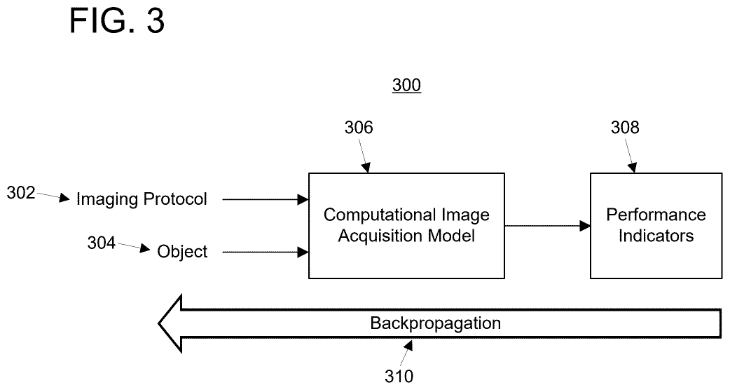

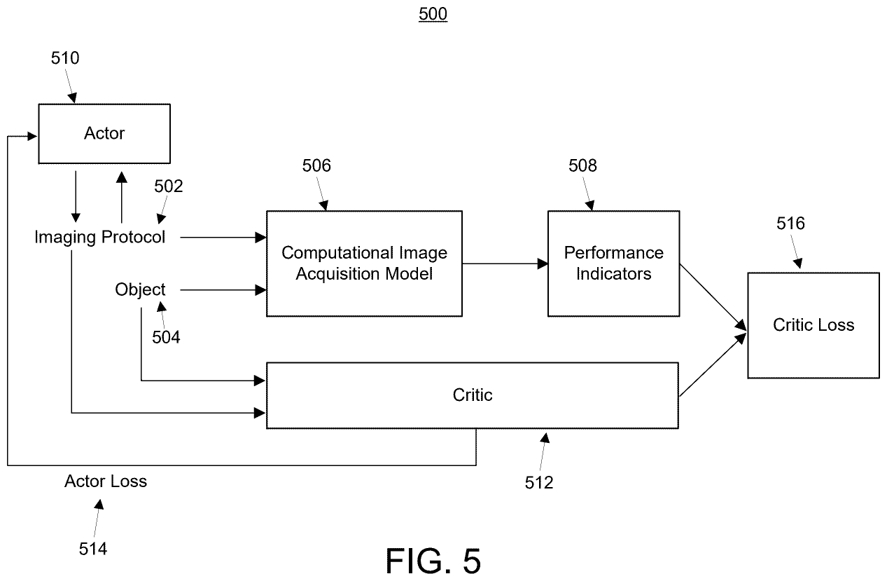

Medical image 206 is then generated from input imaging protocol 202 using computational image acquisition model 204. Computational image acquisition model 204 may model new hardware and/or software features of the target medical image acquisition device. Computational image acquisition model 204 performs Bloch simulation on input imaging protocol 202 to simulate measurements of an image acquisition by the target medical image acquisition device and image reconstruction is performed to generate medical image 206. Medical image 206 is compared with a medical image generated by the reference medical image acquisition device according to input imaging protocol 202 to evaluate and test the new hardware and/or software features. Optimization is then performed to iteratively adjust input imaging protocol 202 based on the comparison to determine the target imaging protocol. The optimization may be performed according to workflow 300 of FIG. 3, workflow 400 of FIG. 4, and/or workflow 500 of FIG. 5, in accordance with one or more embodiments. The target imaging protocol may be determined in workflow 200 of FIG. 2 at step 104 of FIG. 1.

FIG. 3 shows a workflow 300 for optimizing an imaging protocol by differentiable simulation to determine a target imaging protocol, in accordance with one or more embodiments. Workflow 300 of FIG. 3 may be performed during workflow 200 of FIG. 2 for optimizing the imaging parameters to determine the target imaging protocol. In workflow 300, imaging protocol 302 and object 304 are received by computational image acquisition model 306. Imaging protocol 302 is initially the input imaging protocol during the first iteration. Object 304 comprises the necessary information to simulate a new protocol (e.g., T1, T2 and proton density maps). Computational image acquisition model 306 generates a medical image based on imaging protocol 302 and object 304, and performance indicators 308 are determined based on the generated medical images. Backpropagation 310 is then performed for adjusting imaging protocol 302 based on performance indicators 308. The adjusted imaging protocol is then applied as imaging protocol 302 and object 304 is recomputed during the next iteration. Workflow 300 is iteratively repeated to adjust imaging protocol 302 by gradient descent to optimize performance indicators 308 to thereby determine the target imaging protocol. In one embodiment, for example where the goal is to generate a similar image as a reference image, the reference image may also be received as input to determine performance indicators 308.

FIG. 4 shows a workflow 400 for optimizing an imaging protocol by differentiable critic to determine a target imaging protocol, in accordance with one or more embodiments. Workflow 400 of FIG. 4 may be performed during workflow 200 of FIG. 2 for optimizing the imaging parameters to determine the target imaging protocol. In workflow 400, a differentiable critic network 410 is trained to predict performance indicators based on imaging protocol 402 and object 404. Object 404 comprises the necessary information to simulate a new protocol. Imaging protocol 402 is initially the input imaging protocol during the first iteration. Computational image acquisition model 406 generates a medical image based on imaging protocol 402 and object 404, and performance indicators 408 are determined based on the generated medical image. Critic loss 416 is calculated based on performance indicators 408 and the predicted performance indicators predicted by critic network 410. Once critic network 410 is trained, imaging protocol 402 is iteratively adjusted by gradient descent of the output of critic network 410 is performed by backpropagation 414. The adjusted imaging protocol 402 is output as the target imaging protocol.

FIG. 5 shows a workflow 500 for optimizing an imaging protocol by reinforcement learning to determine a target imaging protocol, in accordance with one or more embodiments. Workflow 500 of FIG. 5 may be performed during workflow 200 of FIG. 2 for optimizing the imaging parameters to determine the target imaging protocol. In workflow 500, an actor/critic network is applied. Critic network 512 learns to predict performance indicators based on imaging protocol 502 and object 504. Actor network 510 learns to receive imaging protocol 502 and object 504 as input and output an adjust imaging protocol expected to improve performance indicators 508 according to actor loss 514. Object 504 comprises the necessary information to simulate a new protocol. Imaging protocol 502 is initially the input imaging protocol during the first iteration. Computational image acquisition model 506 generates a medical image based on imaging protocol 502 and object 504, and performance indicators 508 are determined based on the generated medical image. Critic loss 516 is calculated based on performance indicators 508 and the predicted performance indicators predicted by critic network 512. The steps of workflow 500 are iteratively repeated to optimize critic loss 516. The adjusted imaging protocol 502 is output as the target imaging protocol.

In one embodiment, the imaging protocol is optimized to determine the target imaging protocol using a combination of workflow 300 of FIG. 3, workflow 400 of FIG. 4, and/or workflow 500 of FIG. 5. For example, the differentiable critic approach of workflow 500 of FIG. 4 or the reinforcement learning approach of workflow 500 of FIG. 5 may be initially applied. When imaging protocol 402 or 502 is adjusted to be relatively close (as learned during training), the differentiable simulation approach of workflow 300 of FIG. 3 is then applied to determine the target imaging protocol.

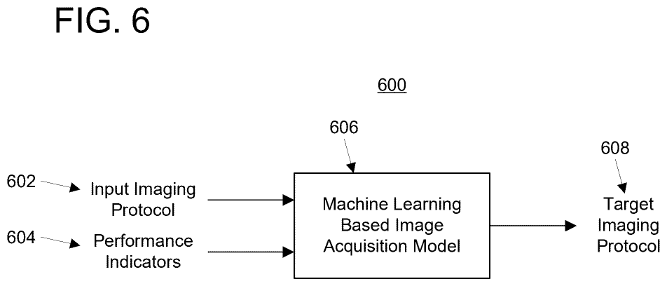

FIG. 6 shows a workflow 600 for determining a target imaging protocol for a target medical image acquisition device from an input imaging protocol for a reference medical image acquisition device using a machine learning based image acquisition model of the target medical image acquisition device, in accordance with one or more embodiments. Workflow 600 may be performed according to method 100 of FIG. 1.

In workflow 600, input imaging protocol 602 and performance indicators 604 are received. Input imaging protocol 602 is for a reference medical image acquisition device of a particular type. Input imaging protocol 602 and performance indicators 604 may be received at step 102 of FIG. 1.

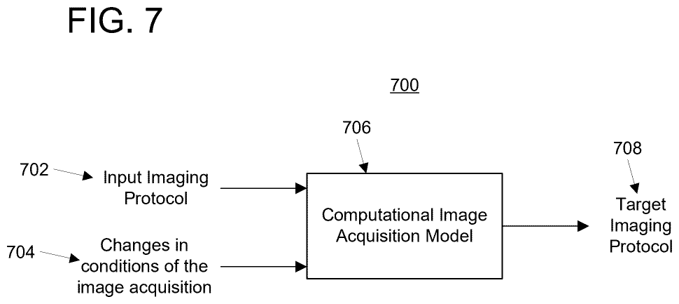

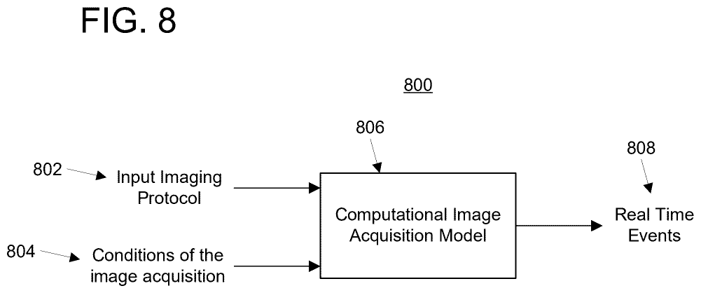

Target imaging protocol 608 is determined from input imaging protocol 602 using machine learning based image acquisition model 606 based on performance indicators 604. Target imaging protocol 608 is for a target medical image acquisition device of a different type. Machine learning based image acquisition model 606 receives input imaging protocol 602 and performance indicators 604 as input and generates as output target imaging protocol 608. Machine learning based image acquisition model 606 is trained to reproduce the results of iterative protocol optimization of a computation image acquisition model (e.g., computation image acquisition model 306 of FIG. 3, 406 of FIG. 4, 506 of FIG. 5, 706 of FIG. 7, or 806 of FIG. 8) in a single shot. Machine learning based image acquisition model 606 reduces the latency to output target imaging protocol 608, e.g., for interactive applications where a user decides to change one of the parameters manually and the protocol must be reoptimized to fit this additional constraint.

FIG. 7 shows a workflow 700 for determining a target imaging protocol for a medical image acquisition device for target conditions of the image acquisition from an input imaging protocol for the medical image acquisition device using a computational image acquisition model of the medical image acquisition device, in accordance with one or more embodiments. Workflow 700 may be performed according to method 100 of FIG. 1.

In workflow 700, input imaging protocol 702 and changes in conditions of the image acquisition 704 are received. Changes in conditions of the image acquisition 704 define changes between the condition of the medical image acquisition device and/or the patient for input imaging protocol 702 and the condition of the same medical image acquisition device and/or the patient for the target imaging protocol 708. Accordingly, input imaging protocol 702 is for initial conditions of the image acquisition. For example, the changes may define a change in a patient size for which input imaging protocol 702 is applicable to a patient size for which target imaging protocol 708 is applicable. Input imaging protocol 702 and changes in conditions of the image acquisition 704 may be received at step 102 of FIG. 1.

Computational image acquisition model 706 is implemented as a computational model, e.g., defined by a set of Bloch equations. Similar to workflow 200 of FIG. 2, computational image acquisition model 706 performs Bloch simulation on input imaging protocol 702 to simulate measurements of an image acquisition by the medical image acquisition device and image reconstruction is performed to generate a medical image. The generated medical image is compared with a medical image generated by the medical image acquisition device according to input imaging protocol 702 to evaluate and test the new hardware and/or software features. Optimization is then performed to tune input imaging protocol 702 based on the comparison to determine target imaging protocol 708. The target imaging protocol is for a target condition of the image acquisition. The optimization may be performed according to workflow 300 of FIG. 3, workflow 400 of FIG. 4, and/or workflow 500 of FIG. 5, in accordance with one or more embodiments. The target imaging protocol may be determined in workflow 700 of FIG. 7 at step 104 of FIG. 1.

FIG. 8 shows a workflow 800 for determining real time events for a medial image acquisition device according to an input imaging protocol and image acquisition conditions, in accordance with one embodiment. Workflow 800 may be performed according to method 100 of FIG. 1.

In workflow 800, input imaging protocol 802 and conditions of the image acquisition 804 are received. Conditions of the image acquisition 804 defines changes of the medical image acquisition device and/or the patient. Input imaging protocol 802 and conditions of the image acquisition 804 may be received at step 102 of FIG. 1.

Computational image acquisition model 806 simulates image acquisition according to input imaging protocol 802 and conditions of the image acquisition 804 to determine real time events 808. Computational image acquisition model 806 may model new hardware and/or software features of the target medical image acquisition device. Computational image acquisition model 806 is implemented as a computational model, e.g., defined by a set of Bloch equations. Computational image acquisition model 806 performs Bloch simulation on input imaging protocol 802 according to conditions of the image acquisition 804 to simulate measurements of an image acquisition by the target medical image acquisition device and determine real time events 808. Such real time events 808 are determined substantially in real time (e.g., <=1 millisecond). Real time events 808 are generated when the user sets up the protocol at the medical image acquisition device. Any parameters that the user changes may require the change of other parameters. For example, adding extra preparation pulses can require increasing TR (repetition time)/TE (echo time) to have enough time to play the pulses, and changing flip angles requires checking that SAR safety limits are still satisfied. Where real time events 808 identifies a bug or error, input imaging protocols may be tuned by optimization, as described above with respect to workflow 200 of FIG. 2.

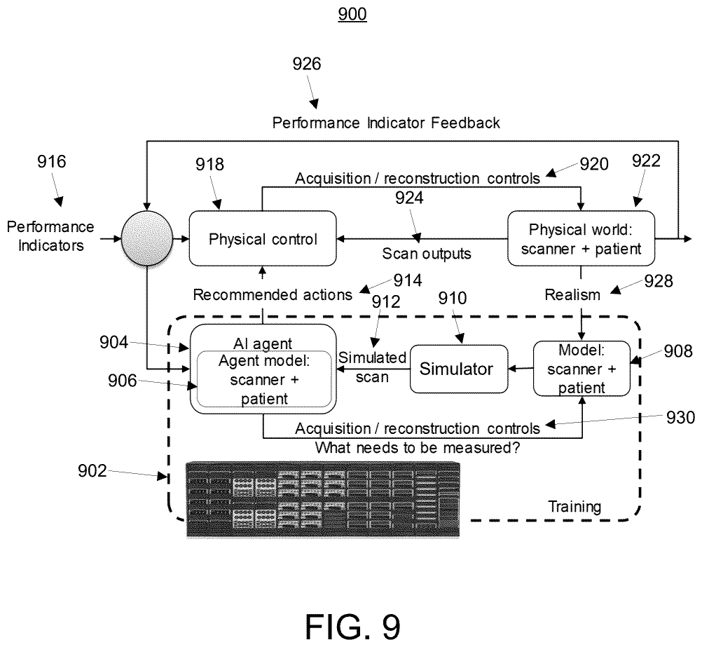

FIG. 9 shows a workflow 900 for training and applying an AI (artificial intelligence) agent 904 for predicting a target imaging protocol, in accordance with one or more embodiments. In some embodiments, AI agent 904 may be critic network 412 of FIG. 4 or machine learning based image acquisition model 606 of FIG. 6.

AI agent 904 is trained during a training stage 902, e.g., on billions of scenarios. AI agent 904 comprises an agent model 906 of the medical image acquisition device and of the patient. AI agent 904 predicts acquisition/reconstruction controls 930 according to performance indicators 916 and performance indicator feedback 926. Acquisition/reconstruction controls 930 represent imaging parameters. Acquisition/reconstruction controls 930 are input to a model 908 of the medical image acquisition device and of the patient and simulated by simulator 910 to generate simulated scan 912. Model 908 comprises information necessary to simulate the evolution of the world. It has complete information about the state of the world but it does not take any decisions. AI agent 904 is the main network of the system. AI agent 904 collects observations about the world and uses them determine which action it should attempt next. In general, AI agent 904 can only access partial observations about the world, so even if AI agent 904 maintains its own world model, that is an incomplete and uncertain model of the agent's beliefs about the world, not the true world model. Simulated scan 912 may be generated similarly to workflow 200 of FIG. 2, where simulator 910 corresponds to computational image acquisition model 204. Model 906 comprises imaging protocols and patient models. AI agent 904 may be trained similarly to FIG. 5: once simulated scan 912 is obtained, performance indicators are determined, then the actor and critic networks of AI agent 904 are updated to generate images with better performance indicators in the future.

Once trained, AI agent 904 is applied to iteratively tune acquisition/reconstruction controls 920 to determine the target imaging protocol. In one example, the target imaging protocol may be determined by AI agent 904 at step 104 of FIG. 1. AI agent 904 is applied to generate recommended actions 914 for physical control 918 of acquisition/reconstruction controls 920 of a physical world scanner (i.e., medical image acquisition device) and patient 922. Physical control 918 is the component that can apply changes to the real world. For example, physical control 918 may be an automated scanner console or a user who reads the recommended actions from AI agent 904 and confirms them for execution at the scanner. Scan outputs 924 are fed back into physical control 918 for optimization. In some embodiments, performance indicators 916 are received as input to physical control 918 for generating acquisition/reconstruction controls 920 of physical work scanner and patient 922. Performance indicators feedback 926 are fed back to physical control 918 for optimization based on performance indicators 916. Performance indicators 916 are the high-level controls that tell the system what should be optimized (e.g., what the tradeoff is between image quality and scan time). Performance indicator feedback 926 is collected for monitoring, e.g., to check that the input performance indicators 916 correspond to desired outcomes.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the image acquisition model utilized at step 104 of FIG. 1, critic network 410 of FIG. 4, actor network 510 and critic network 512 of FIG. 5, machine learning based image acquisition model 606 of FIG. 6, and AI agent 904 of FIG. 9, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

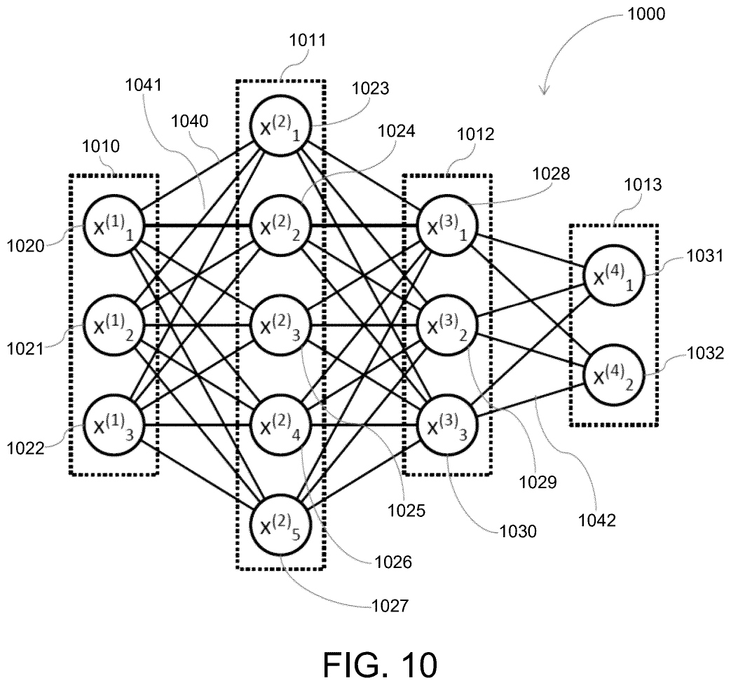

FIG. 10 shows an embodiment of an artificial neural network 1000 that may be used to implement one or more machine learning models described herein. Alternative terms for “artificial neural network” are “neural network”, “artificial neural net” or “neural net”.

The artificial neural network 1000 comprises nodes 1020, . . . , 1032 and edges 1040, . . . , 1042, wherein each edge 1040, . . . , 1042 is a directed connection from a first node 1020, . . . , 1032 to a second node 1020, . . . , 1032. In general, the first node 1020, . . . , 1032 and the second node 1020, . . . , 1032 are different nodes 1020, . . . , 1032, it is also possible that the first node 1020, . . . , 1032 and the second node 1020, . . . , 1032 are identical. For example, in FIG. 10 the edge 1040 is a directed connection from the node 1020 to the node 1023, and the edge 1042 is a directed connection from the node 1030 to the node 1032. An edge 1040, . . . , 1042 from a first node 1020, . . . , 1032 to a second node 1020, . . . , 1032 is also denoted as “ingoing edge” for the second node 1020, . . . , 1032 and as “outgoing edge” for the first node 1020, . . . , 1032.

In this embodiment, the nodes 1020, . . . , 1032 of the artificial neural network 1000 can be arranged in layers 1010, . . . , 1013, wherein the layers can comprise an intrinsic order introduced by the edges 1040, . . . , 1042 between the nodes 1020, . . . , 1032.

In particular, edges 1040, . . . , 1042 can exist only between neighboring layers of nodes. In the displayed embodiment, there is an input layer 1010 comprising only nodes 1020, . . . , 1022 without an incoming edge, an output layer 1013 comprising only nodes 1031, 1032 without outgoing edges, and hidden layers 1011, 1012 in-between the input layer 1010 and the output layer 1013. In general, the number of hidden layers 1011, 1012 can be chosen arbitrarily. The number of nodes 1020, . . . , 1022 within the input layer 1010 usually relates to the number of input values of the neural network, and the number of nodes 1031, 1032 within the output layer 1013 usually relates to the number of output values of the neural network.

In particular, a (real) number can be assigned as a value to every node 1020, . . . , 1032 of the neural network 1000. Here, x⁽ⁿ⁾ᵢ denotes the value of the i-th node 1020, . . . , 1032 of the n-th layer 1010, . . . , 1013. The values of the nodes 1020, . . . , 1022 of the input layer 1010 are equivalent to the input values of the neural network 1000, the values of the nodes 1031, 1032 of the output layer 1013 are equivalent to the output value of the neural network 1000. Furthermore, each edge 1040, . . . , 1042 can comprise a weight being a real number, in particular, the weight is a real number within the interval [−1, 1] or within the interval [0, 1]. Here, w^{(m,n)}ᵢⱼ denotes the weight of the edge between the i-th node 1020, . . . , 1032 of the m-th layer 1010, . . . , 1013 and the j-th node 1020, . . . , 1032 of the n-th layer 1010, . . . , 1013. Furthermore, the abbreviation w⁽ⁿ⁾_{i,j} is defined for the weight

$${w}_{i , j}^{( n , n + 1 )} .$$

In particular, to calculate the output values of the neural network 1000, the input values are propagated through the neural network. In particular, the values of the nodes 1020, . . . , 1032 of the (n+1)-th layer 1010, . . . , 1013 can be calculated based on the values of the nodes 1020, . . . , 1032 of the n-th layer 1010, . . . , 1013 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 1010 are given by the input of the neural network 1000, wherein values of the first hid-den layer 1011 can be calculated based on the values of the input layer 1010 of the neural network, wherein values of the second hidden layer 1012 can be calculated based in the values of the first hidden layer 1011, etc.

In order to set the values w^{(m,n)}_{i,j} for the edges, the neural network 1000 has to be trained using training data. In particular, training data comprises training input data and training output data (denoted as ti). For a training step, the neural network 1000 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 1000 (backpropagation algorithm). In particular, the weights are changed according to

$${w}^{′ {( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 1013, wherein f′ is the first derivative of the activation function, and t⁽ⁿ⁺¹⁾ⱼ is the comparison training value for the j-th node of the output layer 1013.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

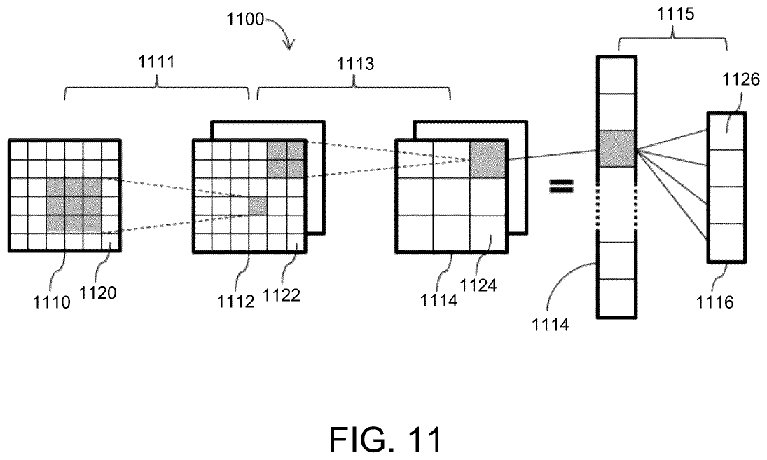

FIG. 11 shows an embodiment of a convolutional neural network 1100 that may be used to implement one or more machine learning models described herein. In the displayed embodiment, the convolutional neural network comprises 1100 an input node layer 1110, a convolutional layer 1111, a pooling layer 1113, a fully connected layer 1114 and an output node layer 1116, as well as hidden node layers 1112, 1114. Alternatively, the convolutional neural network 1100 can comprise several convolutional layers 1111, several pooling layers 1113 and several fully connected layers 1115, as well as other types of layers. The order of the layers can be chosen arbitrarily, usually fully connected layers 1115 are used as the last layers before the output layer 1116.

In particular, within a convolutional neural network 1100 nodes 1120, 1122, 1124 of a node layer 1110, 1112, 1114 can be considered to be arranged as a d-dimensional matrix or as a d-dimensional image. In particular, in the two-dimensional case the value of the node 1120, 1122, 1124 indexed with i and j in the n-th node layer 1110, 1112, 1114 can be denoted as x(n)[i, j]. However, the arrangement of the nodes 1120, 1122, 1124 of one node layer 1110, 1112, 1114 does not have an effect on the calculations executed within the convolutional neural network 1100 as such, since these are given solely by the structure and the weights of the edges.

A convolutional layer 1111 is a connection layer between an anterior node layer 1110 (with node values x(n−1)) and a posterior node layer 1112 (with node values x(n)). In particular, a convolutional layer 1111 is characterized by the structure and the weights of the incoming edges forming a convolution operation based on a certain number of kernels. In particular, the structure and the weights of the edges of the convolutional layer 1111 are chosen such that the values x(n) of the nodes 1122 of the posterior node layer 1112 are calculated as a convolution x(n)=K*x(n−1) based on the values x(n−1) of the nodes 1120 anterior node layer 1110, where the convolution * is defined in the two-dimensional case as

$${x}_{k}^{( n )} [ i , j ] = ( {\begin{matrix}K & {}^{*} {x}^{( n - 1 )} ) [ i , j ] = \sum \end{matrix}}_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 1120, 1122 (e.g., a 3×3 matrix, or a 5×5 matrix). In particular, this implies that the weights of the edges in the convolution layer 1111 are not independent, but chosen such that they produce said convolution equation. In particular, for a kernel being a 3×3 matrix, there are only 9 independent weights (each entry of the kernel matrix corresponding to one independent weight), irrespectively of the number of nodes 1120, 1122 in the anterior node layer 1110 and the posterior node layer 1112.

In general, convolutional neural networks 1100 use node layers 1110, 1112, 1114 with a plurality of channels, in particular, due to the use of a plurality of kernels in convolutional layers 1111. In those cases, the node layers can be considered as (d+1)-dimensional matrices (the first dimension indexing the channels). The action of a convolutional layer 1111 is then a two-dimensional example defined as

$${x}^{{( n )}_{b}} [ i , j ] = {\sum }_{a} {K}_{a , b} {}^{*} {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ₐ corresponds to the a-th channel of the anterior node layer 1110, x⁽ⁿ⁾_{b} corresponds to the b-th channel of the posterior node layer 1112 and K_{a,b} corresponds to one of the kernels. If a convolutional layer 1111 acts on an anterior node layer 1110 with A channels and outputs a posterior node layer 1112 with B channels, there are A·B independent d-dimensional kernels K_{a,b}.

In general, in convolutional neural networks 1100 activation functions are used. In this embodiment re ReLU (acronym for “Rectified Linear Units”) is used, with R(z)=max(0, z), so that the action of the convolutional layer 1111 in the two-dimensional example is

$${x}^{{( n )}_{b}} [ i , j ] = R ( {\sum }_{a} {K}_{a , b} {}^{*} {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 1110 comprises 36 nodes 1120, arranged as a two-dimensional 6×6 matrix. The first hidden node layer 1112 comprises 72 nodes 1122, arranged as two two-dimensional 6×6 matrices, each of the two matrices being the result of a convolution of the values of the input layer with a 3×3 kernel within the convolutional layer 1111. Equivalently, the nodes 1122 of the first hidden node layer 1112 can be interpreted as arranged as a three-dimensional 2×6×6 matrix, wherein the first dimension correspond to the channel dimension.

The advantage of using convolutional layers 1111 is that spatially local correlation of the input data can exploited by enforcing a local connectivity pattern between nodes of adjacent layers, in particular by each node being connected to only a small region of the nodes of the preceding layer.

A pooling layer 1113 is a connection layer between an anterior node layer 1112 (with node values x(n−1)) and a posterior node layer 1114 (with node values x(n)). In particular, a pooling layer 1113 can be characterized by the structure and the weights of the edges and the activation function forming a pooling operation based on a non-linear pooling function f. For example, in the two-dimensional case the values x(n) of the nodes 1124 of the posterior node layer 1114 can be calculated based on the values x(n−1) of the nodes 1122 of the anterior node layer 1112 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 1113 the number of nodes 1122, 1124 can be reduced, by re-placing a number d1·d2 of neighboring nodes 1122 in the anterior node layer 1112 with a single node 1122 in the posterior node layer 1114 being calculated as a function of the values of said number of neighboring nodes. In particular, the pooling function f can be the max-function, the average or the L2-Norm. In particular, for a pooling layer 1113 the weights of the incoming edges are fixed and are not modified by training.

The advantage of using a pooling layer 1113 is that the number of nodes 1122, 1124 and the number of parameters is reduced. This leads to the amount of computation in the network being reduced and to a control of overfitting.

In the displayed embodiment, the pooling layer 1113 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 1100 are fully connected layers 1115. A fully connected layer 1115 is a connection layer between an anterior node layer 1114 and a posterior node layer 1116. A fully connected layer 1113 can be characterized by the fact that a majority, in particular, all edges between nodes 1114 of the anterior node layer 1114 and the nodes 1116 of the posterior node layer are present, and wherein the weight of each of these edges can be adjusted individually.

In this embodiment, the nodes 1124 of the anterior node layer 1114 of the fully connected layer 1115 are displayed both as two-dimensional matrices, and additionally as non-related nodes (indicated as a line of nodes, wherein the number of nodes was reduced for a better presentability). This operation is also denoted as “flattening”. In this embodiment, the number of nodes 1126 in the posterior node layer 1116 of the fully connected layer 1115 smaller than the number of nodes 1124 in the anterior node layer 1114. Alternatively, the number of nodes 1126 can be equal or larger.

Furthermore, in this embodiment the Softmax activation function is used within the fully connected layer 1115. By applying the Softmax function, the sum the values of all nodes 1126 of the output layer 1116 is 1, and all values of all nodes 1126 of the output layer 1116 are real numbers between 0 and 1. In particular, if using the convolutional neural network 1100 for categorizing input data, the values of the output layer 1116 can be interpreted as the probability of the input data falling into one of the different categories.

In particular, convolutional neural networks 1100 can be trained based on the backpropagation algorithm. For preventing overfitting, methods of regularization can be used, e.g., dropout of nodes 1120, . . . , 1124, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

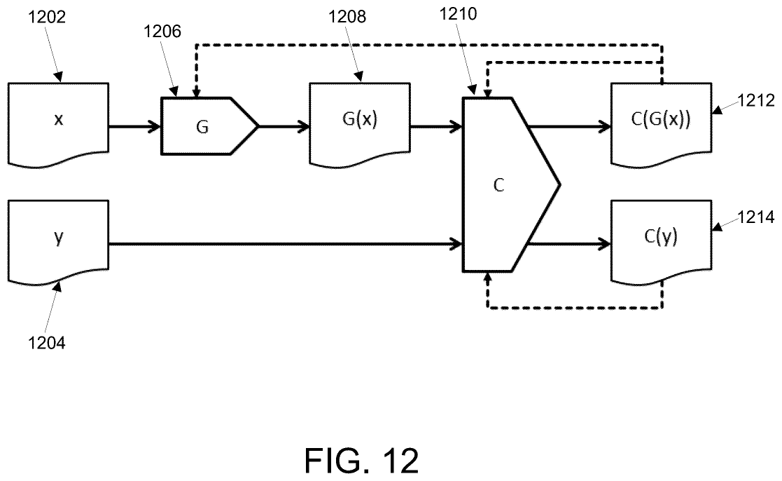

FIG. 12 shows a data flow diagram according to an embodiment for using a generative adversarial network for creating synthetic output data G(x) 1208 based on input data x 1202 that is indistinguishable from real output data y 1204, in accordance with one or more embodiments. The synthetic output data G(x) 1208 has the same structure as the real output data y 1204, but its content is not derived from real world data.

The generative adversarial network comprises a generator function G 1206 and a classifier function C 1210 which are trained jointly. The task of the generator function G 1206 is to provide realistic synthetic output data G(x) 1208 based on input data x 1202, and the task of the classifier function C 1210 is to distinguish between real output data y 1204 and synthetic output data G(x) 1208. In particular, the output of the classifier function C 1210 is a real number between 0 and 1 corresponding to the probability of the input value being real data, so that an ideal classifier function would calculate an output value of C(y) 1214≈1 for real data y 1204 and C(G(x)) 1212≈0 for synthetic data G(x) 1208.

Within the training process, parameters of the generator function G 1206 are adapted so that the synthetic output data G(x) 1208 has the same characteristics as real output data y 1204, so that the classifier function C 1210 cannot distinguish between real and synthetic data anymore. At the same time, parameters of the classifier function C 1210 are adapted so that it distinguishes between real and synthetic data in the best possible way. Here, the training relies on pairs comprising input data x 1202 and the corresponding real output data y 1204. Within a single training step, the generator function G 1206 is applied to the input data x 1202 for generating synthetic output data G(x) 1208. Furthermore, the classifier function C 1210 is applied to the real output data y 1204 for generating a first classification result C(y) 1214. Additionally, the classifier function C 1210 is applied to the synthetic output data G(x) 1208 for generating a second classification result C(G(x)) 1212.

Adapting the parameters of the generative function G 1206 and the classifier function C 1210 is based on minimizing a cost function by using the backpropagation algorithm, respectively. In this embodiment, the cost function K_{C} for the classifier function C 1210 is K_{C}∝−BCE(C(y), 1)−BCE(C(G(x), 0), wherein BCE denotes the binary cross entropy defined as BCE(z, z′)=z′·log(z)+(1−z′)·log(1−z). By using this cost function, both wrongly classifying real output data as synthetic (indicated by C(y)≈0) and wrongly classifying synthetic output data as real (indicated as C(G(x)) 1212≈1) increases the cost function K_{C} to be minimized. Furthermore, the cost function K_{G} for the generator function G 1206 is K_{G} ∝−BCE(C(G(x), 1)=−log(C(G(x)). By using this cost function, correctly classified synthetic output data (indicated as C(G(x)) 1212≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

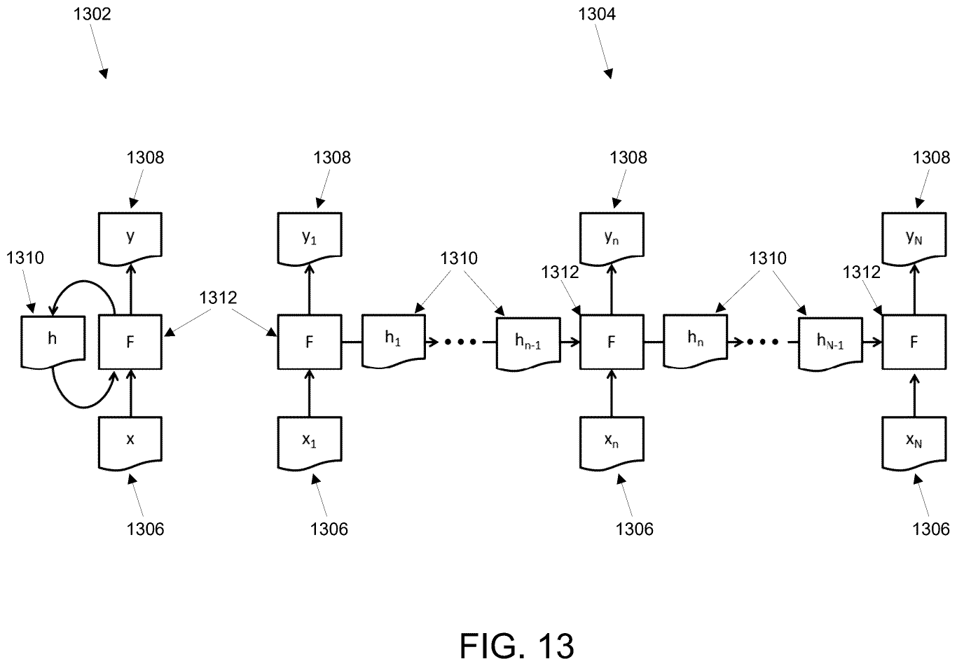

FIG. 13 shows the schematic structure of a recurrent machine learning model F, both in a recurrent representation 1302 and in an unfolded representation 1304, that may be used to implement one or more machine learning models described herein. The recurrent machine learning model takes as input several input datasets x, x₁, . . . , x_{N} 1306 and creates a corresponding set of output datasets y, y₁, . . . , y_{N} 1308. Furthermore, the output depends on a so-called hidden vector h, h₁, . . . , h_{N} 1310, which implicitly comprises information about input datasets previously used as input for the recurrent machine learning model F 1312. By using these hidden vectors h, h₁, . . . , h_{N} 1310, a sequentiality of the input datasets can be leveraged.

In a single step of the processing, the recurrent machine learning model F 1312 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 1312 into a part F(y) calculating the output data and F(h) calculating the hidden vector, one step of processing calculates yₙ=F^{(y)}(xₙ, hⁿ⁻¹) and hₙ=F^{(h)}(xₙ, hₙ₋₁). For the first processing step, h₀ can be chosen randomly or filled with all entries being zero. The parameters of the recurrent machine learning model F 1312 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-9. Certain steps or functions of the methods and workflows described herein, including one or more of the steps or functions of FIGS. 1-9, may be performed by a server or by another processor in a network-based cloud-computing system. Certain steps or functions of the methods and workflows described herein, including one or more of the steps of FIGS. 1-9, may be performed by a client computer in a network-based cloud computing system. The steps or functions of the methods and workflows described herein, including one or more of the steps of FIGS. 1-9, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-9, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1402 that may be used to implement systems, apparatuses, and methods described herein is depicted in FIG. 14. Computer 1402 includes a processor 1404 operatively coupled to a data storage device 1412 and a memory 1410. Processor 1404 controls the overall operation of computer 1402 by executing computer program instructions that define such operations. The computer program instructions may be stored in data storage device 1412, or other computer readable medium, and loaded into memory 1410 when execution of the computer program instructions is desired. Thus, the method and workflow steps or functions of FIGS. 1-9 can be defined by the computer program instructions stored in memory 1410 and/or data storage device 1412 and controlled by processor 1404 executing the computer program instructions. For example, the computer program instructions can be implemented as computer executable code programmed by one skilled in the art to perform the method and workflow steps or functions of FIGS. 1-9. Accordingly, by executing the computer program instructions, the processor 1404 executes the method and workflow steps or functions of FIGS. 1-9. Computer 1402 may also include one or more network interfaces 1406 for communicating with other devices via a network. Computer 1402 may also include one or more input/output devices 1408 that enable user interaction with computer 1402 (e.g., display, keyboard, mouse, speakers, buttons, etc.).

Processor 1404 may include both general and special purpose microprocessors, and may be the sole processor or one of multiple processors of computer 1402. Processor 1404 may include one or more central processing units (CPUs), for example. Processor 1404, data storage device 1412, and/or memory 1410 may include, be supplemented by, or incorporated in, one or more application-specific integrated circuits (ASICs) and/or one or more field programmable gate arrays (FPGAs).

Data storage device 1412 and memory 1410 each include a tangible non-transitory computer readable storage medium. Data storage device 1412, and memory 1410, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1408 may include peripherals, such as a printer, scanner, display screen, etc. For example, input/output devices 1408 may include a display device such as a cathode ray tube (CRT) or liquid crystal display (LCD) monitor for displaying information to the user, a keyboard, and a pointing device such as a mouse or a trackball by which the user can provide input to computer 1402.

An image acquisition device 1414 can be connected to the computer 1402 to input image data (e.g., medical images) to the computer 1402. It is possible to implement the image acquisition device 1414 and the computer 1402 as one device. It is also possible that the image acquisition device 1414 and the computer 1402 communicate wirelessly through a network. In a possible embodiment, the computer 1402 can be located remotely with respect to the image acquisition device 1414.

Any or all of the systems, apparatuses, and methods discussed herein may be implemented using one or more computers such as computer 1402.

One skilled in the art will recognize that an implementation of an actual computer or computer system may have other structures and may contain other components as well, and that FIG. 14 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving at least one of 1) one or more performance indicators, 2) an input imaging protocol for an image acquisition, or 3) changes in conditions of the image acquisition; determining a target imaging protocol using an image acquisition model based on the at least one of the one or more performance indicators, the input imaging protocol, or the changes in the conditions of the image acquisition; and outputting the target imaging protocol.

Illustrative embodiment 2. The computer-implemented method of illustrative embodiment 1, wherein: receiving at least one of 1) one or more performance indicators, 2) an input imaging protocol for an image acquisition, or 3) changes in conditions of the image acquisition comprises: receiving the input imaging protocol, the input imaging protocol being for a reference medical image acquisition device; and determining a target imaging protocol using an image acquisition model based on the at least one of the one or more performance indicators, the input imaging protocol, or the changes in the conditions of the image acquisition comprises: generating an image using a computation image acquisition model based on the input imaging protocol, comparing the generated image with a medical image generated by the reference medical image acquisition device, and iteratively adjusting the input imaging protocol based on the comparison to determine the target imaging protocol, the target imaging protocol being for a target medical image acquisition device.

Illustrative embodiment 3. The computer-implemented method of any one of illustrative embodiments 1-2, wherein iteratively adjusting the input imaging protocol based on the comparison to determine the target imaging protocol comprises: iteratively adjusting the input imaging protocol by backpropagation.

Illustrative embodiment 4. The computer-implemented method of any one of illustrative embodiments 1-3, wherein iteratively adjusting the input imaging protocol based on the comparison to determine the target imaging protocol comprises: iteratively adjusting the input imaging protocol by gradient descent on an output of a critic network, the critic network trained to predict the one or more performance indicators.

Illustrative embodiment 5. The computer-implemented method of any one of illustrative embodiments 1-4, wherein iteratively adjusting the input imaging protocol based on the comparison to determine the target imaging protocol comprises: iteratively adjusting the input imaging protocol by an actor network to optimize a critic loss, the critic loss calculated based on 1) the one or more performance indicators determined based on the comparison and 2) the one or more performance indicators predicted by a critic network.

Illustrative embodiment 6. The computer-implemented method of any one of illustrative embodiments 1-5, wherein: receiving at least one of 1) one or more performance indicators, 2) an input imaging protocol for an image acquisition, or 3) changes in conditions of the image acquisition comprises: receiving the input imaging protocol and the one or more performance indicators, the input imaging protocol being for a reference medical image acquisition device; and determining a target imaging protocol using an image acquisition model based on the at least one of the one or more performance indicators, the input imaging protocol, or the changes in the conditions of the image acquisition comprises: determining the target imaging protocol using a machine learning based image acquisition model, the machine learning based image acquisition model receives as input the input imaging protocol and the one or more performance indicators and generating as output the target imaging protocol, the target imaging protocol being for a target medical image acquisition device.

Illustrative embodiment 7. The computer-implemented method of any one of illustrative embodiments 1-6, wherein: receiving at least one of 1) one or more performance indicators, 2) an input imaging protocol for an image acquisition, or 3) changes in conditions of the image acquisition comprises: receiving the input imaging protocol and the changes in the conditions of the image acquisition, the input imaging protocol being for an initial condition of the image acquisition; and determining a target imaging protocol using an image acquisition model based on the at least one of the one or more performance indicators, the input imaging protocol, or the changes in the conditions of the image acquisition comprises: determining the target imaging protocol using a computational image acquisition model based on the input imaging protocol and the changes in the conditions of the image acquisition, the target imaging protocol being for a target condition of the image acquisition.

Illustrative embodiment 8. The computer-implemented method of any one of illustrative embodiments 1-7, wherein: receiving at least one of 1) one or more performance indicators, 2) an input imaging protocol for an image acquisition, or 3) changes in conditions of the image acquisition comprises: receiving the input imaging protocol and the changes in the conditions of the image acquisition; and determining a target imaging protocol using an image acquisition model based on the at least one of the one or more performance indicators, the input imaging protocol, or the changes in the conditions of the image acquisition comprises: determining events using a computational image acquisition model based on the input imaging protocol and the changes in the conditions of the image acquisition.

Illustrative embodiment 9. The computer-implemented method of any one of illustrative embodiments 8, wherein determining a target imaging protocol using an image acquisition model based on the at least one of the one or more performance indicators, the input imaging protocol, or the changes in the conditions of the image acquisition further comprises: determining the target imaging protocol based on the events.

Illustrative embodiment 10. An apparatus comprising: means for receiving at least one of 1) one or more performance indicators, 2) an input imaging protocol for an image acquisition, or 3) changes in conditions of the image acquisition; means for determining a target imaging protocol using an image acquisition model based on the at least one of the one or more performance indicators, the input imaging protocol, or the changes in the conditions of the image acquisition; and means for outputting the target imaging protocol.

Illustrative embodiment 11. The apparatus of illustrative embodiment 10, wherein: the means for receiving at least one of 1) one or more performance indicators, 2) an input imaging protocol for an image acquisition, or 3) changes in conditions of the image acquisition comprises: means for receiving the input imaging protocol, the input imaging protocol being for a reference medical image acquisition device; and the means for determining a target imaging protocol using an image acquisition model based on the at least one of the one or more performance indicators, the input imaging protocol, or the changes in the conditions of the image acquisition comprises: means for generating an image using a computation image acquisition model based on the input imaging protocol, means for comparing the generated image with a medical image generated by the reference medical image acquisition device, and means for iteratively adjusting the input imaging protocol based on the comparison to determine the target imaging protocol, the target imaging protocol being for a target medical image acquisition device.

Illustrative embodiment 12. The apparatus of any one of illustrative embodiments 10-11, wherein the means for iteratively adjusting the input imaging protocol based on the comparison to determine the target imaging protocol comprises: means for iteratively adjusting the input imaging protocol by backpropagation.

Illustrative embodiment 13. The apparatus of any one of illustrative embodiments 10-12, wherein the means for iteratively adjusting the input imaging protocol based on the comparison to determine the target imaging protocol comprises: means for iteratively adjusting the input imaging protocol by gradient descent on an output of a critic network, the critic network trained to predict the one or more performance indicators.

Illustrative embodiment 14. The apparatus of any one of illustrative embodiments 10-13, wherein the means for iteratively adjusting the input imaging protocol based on the comparison to determine the target imaging protocol comprises: means for iteratively adjusting the input imaging protocol by an actor network to optimize a critic loss, the critic loss calculated based on 1) the one or more performance indicators determined based on the comparison and 2) the one or more performance indicators predicted by a critic network.

Illustrative embodiment 15. A non-transitory computer-readable storage medium comprising instructions which, when executed by a computer, cause the computer to carry out operations comprising: receiving at least one of 1) one or more performance indicators, 2) an input imaging protocol for an image acquisition, or 3) changes in conditions of the image acquisition; determining a target imaging protocol using an image acquisition model based on the at least one of the one or more performance indicators, the input imaging protocol, or the changes in the conditions of the image acquisition; and outputting the target imaging protocol.

Illustrative embodiment 16. The non-transitory computer-readable storage medium of illustrative embodiment 15, wherein: receiving at least one of 1) one or more performance indicators, 2) an input imaging protocol for an image acquisition, or 3) changes in conditions of the image acquisition comprises: receiving the input imaging protocol, the input imaging protocol being for a reference medical image acquisition device; and determining a target imaging protocol using an image acquisition model based on the at least one of the one or more performance indicators, the input imaging protocol, or the changes in the conditions of the image acquisition comprises: generating an image using a computation image acquisition model based on the input imaging protocol, comparing the generated image with a medical image generated by the reference medical image acquisition device, and iteratively adjusting the input imaging protocol based on the comparison to determine the target imaging protocol, the target imaging protocol being for a target medical image acquisition device.

Illustrative embodiment 17. The non-transitory computer-readable storage medium of any one of illustrative embodiments 15-16, wherein: receiving at least one of 1) one or more performance indicators, 2) an input imaging protocol for an image acquisition, or 3) changes in conditions of the image acquisition comprises: receiving the input imaging protocol and the one or more performance indicators, the input imaging protocol being for a reference medical image acquisition device; and determining a target imaging protocol using an image acquisition model based on the at least one of the one or more performance indicators, the input imaging protocol, or the changes in the conditions of the image acquisition comprises: determining the target imaging protocol using a machine learning based image acquisition model, the machine learning based image acquisition model receives as input the input imaging protocol and the one or more performance indicators and generating as output the target imaging protocol, the target imaging protocol being for a target medical image acquisition device.

Illustrative embodiment 18. The non-transitory computer-readable storage medium of any one of illustrative embodiments 15-17, wherein: receiving at least one of 1) one or more performance indicators, 2) an input imaging protocol for an image acquisition, or 3) changes in conditions of the image acquisition comprises: receiving the input imaging protocol and the changes in the conditions of the image acquisition, the input imaging protocol being for an initial condition of the image acquisition; and determining a target imaging protocol using an image acquisition model based on the at least one of the one or more performance indicators, the input imaging protocol, or the changes in the conditions of the image acquisition comprises: determining the target imaging protocol using a computational image acquisition model based on the input imaging protocol and the changes in the conditions of the image acquisition, the target imaging protocol being for a target condition of the image acquisition.

Illustrative embodiment 19. The non-transitory computer-readable storage medium of any one of illustrative embodiments 15-18, wherein: receiving at least one of 1) one or more performance indicators, 2) an input imaging protocol for an image acquisition, or 3) changes in conditions of the image acquisition comprises: receiving the input imaging protocol and the changes in the conditions of the image acquisition; and determining a target imaging protocol using an image acquisition model based on the at least one of the one or more performance indicators, the input imaging protocol, or the changes in the conditions of the image acquisition comprises: determining events using a computational image acquisition model based on the input imaging protocol and the changes in the conditions of the image acquisition.

Illustrative embodiment 20. The non-transitory computer-readable storage medium of any one of illustrative embodiments 19, wherein determining a target imaging protocol using an image acquisition model based on the at least one of the one or more performance indicators, the input imaging protocol, or the changes in the conditions of the image acquisition further comprises: determining the target imaging protocol based on the events.

1. A computer-implemented method comprising:
receiving at least one of 1) one or more performance indicators, 2) an input imaging protocol for an image acquisition, or 3) changes in conditions of the image acquisition;
determining a target imaging protocol using an image acquisition model based on the at least one of the one or more performance indicators, the input imaging protocol, or the changes in the conditions of the image acquisition; and
outputting the target imaging protocol.

2. The computer-implemented method of claim 1, wherein:
receiving at least one of 1) one or more performance indicators, 2) an input imaging protocol for an image acquisition, or 3) changes in conditions of the image acquisition comprises:
receiving the input imaging protocol, the input imaging protocol being for a reference medical image acquisition device; and
determining a target imaging protocol using an image acquisition model based on the at least one of the one or more performance indicators, the input imaging protocol, or the changes in the conditions of the image acquisition comprises:
generating an image using a computation image acquisition model based on the input imaging protocol,
comparing the generated image with a medical image generated by the reference medical image acquisition device, and
iteratively adjusting the input imaging protocol based on the comparison to determine the target imaging protocol, the target imaging protocol being for a target medical image acquisition device.

3. The computer-implemented method of claim 2, wherein iteratively adjusting the input imaging protocol based on the comparison to determine the target imaging protocol comprises:
iteratively adjusting the input imaging protocol by backpropagation.

4. The computer-implemented method of claim 2, wherein iteratively adjusting the input imaging protocol based on the comparison to determine the target imaging protocol comprises:
iteratively adjusting the input imaging protocol by gradient descent on an output of a critic network, the critic network trained to predict the one or more performance indicators.

5. The computer-implemented method of claim 2, wherein iteratively adjusting the input imaging protocol based on the comparison to determine the target imaging protocol comprises:
iteratively adjusting the input imaging protocol by an actor network to optimize a critic loss, the critic loss calculated based on 1) the one or more performance indicators determined based on the comparison and 2) the one or more performance indicators predicted by a critic network.

6. The computer-implemented method of claim 1, wherein:
receiving at least one of 1) one or more performance indicators, 2) an input imaging protocol for an image acquisition, or 3) changes in conditions of the image acquisition comprises:
receiving the input imaging protocol and the one or more performance indicators, the input imaging protocol being for a reference medical image acquisition device; and
determining a target imaging protocol using an image acquisition model based on the at least one of the one or more performance indicators, the input imaging protocol, or the changes in the conditions of the image acquisition comprises:
determining the target imaging protocol using a machine learning based image acquisition model, the machine learning based image acquisition model receives as input the input imaging protocol and the one or more performance indicators and generating as output the target imaging protocol, the target imaging protocol being for a target medical image acquisition device.

7. The computer-implemented method of claim 1, wherein:
receiving at least one of 1) one or more performance indicators, 2) an input imaging protocol for an image acquisition, or 3) changes in conditions of the image acquisition comprises:
receiving the input imaging protocol and the changes in the conditions of the image acquisition, the input imaging protocol being for an initial condition of the image acquisition; and
determining a target imaging protocol using an image acquisition model based on the at least one of the one or more performance indicators, the input imaging protocol, or the changes in the conditions of the image acquisition comprises:
determining the target imaging protocol using a computational image acquisition model based on the input imaging protocol and the changes in the conditions of the image acquisition, the target imaging protocol being for a target condition of the image acquisition.

8. The computer-implemented method of claim 1, wherein:
receiving at least one of 1) one or more performance indicators, 2) an input imaging protocol for an image acquisition, or 3) changes in conditions of the image acquisition comprises:
receiving the input imaging protocol and the changes in the conditions of the image acquisition; and
determining a target imaging protocol using an image acquisition model based on the at least one of the one or more performance indicators, the input imaging protocol, or the changes in the conditions of the image acquisition comprises:
determining events using a computational image acquisition model based on the input imaging protocol and the changes in the conditions of the image acquisition.

9. The computer-implemented method of claim 8, wherein determining a target imaging protocol using an image acquisition model based on the at least one of the one or more performance indicators, the input imaging protocol, or the changes in the conditions of the image acquisition further comprises:
determining the target imaging protocol based on the events.

10. An apparatus comprising:
means for receiving at least one of 1) one or more performance indicators, 2) an input imaging protocol for an image acquisition, or 3) changes in conditions of the image acquisition;
means for determining a target imaging protocol using an image acquisition model based on the at least one of the one or more performance indicators, the input imaging protocol, or the changes in the conditions of the image acquisition; and
means for outputting the target imaging protocol.

11. The apparatus of claim 10, wherein:
the means for receiving at least one of 1) one or more performance indicators, 2) an input imaging protocol for an image acquisition, or 3) changes in conditions of the image acquisition comprises:
means for receiving the input imaging protocol, the input imaging protocol being for a reference medical image acquisition device; and
the means for determining a target imaging protocol using an image acquisition model based on the at least one of the one or more performance indicators, the input imaging protocol, or the changes in the conditions of the image acquisition comprises:
means for generating an image using a computation image acquisition model based on the input imaging protocol,
means for comparing the generated image with a medical image generated by the reference medical image acquisition device, and
means for iteratively adjusting the input imaging protocol based on the comparison to determine the target imaging protocol, the target imaging protocol being for a target medical image acquisition device.

12. The apparatus of claim 11, wherein the means for iteratively adjusting the input imaging protocol based on the comparison to determine the target imaging protocol comprises:
means for iteratively adjusting the input imaging protocol by backpropagation.

13. The apparatus of claim 11, wherein the means for iteratively adjusting the input imaging protocol based on the comparison to determine the target imaging protocol comprises:
means for iteratively adjusting the input imaging protocol by gradient descent on an output of a critic network, the critic network trained to predict the one or more performance indicators.

14. The apparatus of claim 11, wherein the means for iteratively adjusting the input imaging protocol based on the comparison to determine the target imaging protocol comprises:
means for iteratively adjusting the input imaging protocol by an actor network to optimize a critic loss, the critic loss calculated based on 1) the one or more performance indicators determined based on the comparison and 2) the one or more performance indicators predicted by a critic network.

15. A non-transitory computer-readable storage medium comprising instructions which, when executed by a computer, cause the computer to carry out operations comprising:
receiving at least one of 1) one or more performance indicators, 2) an input imaging protocol for an image acquisition, or 3) changes in conditions of the image acquisition;
determining a target imaging protocol using an image acquisition model based on the at least one of the one or more performance indicators, the input imaging protocol, or the changes in the conditions of the image acquisition; and
outputting the target imaging protocol.

16. The non-transitory computer-readable storage medium of claim 15, wherein:
receiving at least one of 1) one or more performance indicators, 2) an input imaging protocol for an image acquisition, or 3) changes in conditions of the image acquisition comprises:
receiving the input imaging protocol, the input imaging protocol being for a reference medical image acquisition device; and
determining a target imaging protocol using an image acquisition model based on the at least one of the one or more performance indicators, the input imaging protocol, or the changes in the conditions of the image acquisition comprises:
generating an image using a computation image acquisition model based on the input imaging protocol,
comparing the generated image with a medical image generated by the reference medical image acquisition device, and
iteratively adjusting the input imaging protocol based on the comparison to determine the target imaging protocol, the target imaging protocol being for a target medical image acquisition device.

17. The non-transitory computer-readable storage medium of claim 15, wherein:
receiving at least one of 1) one or more performance indicators, 2) an input imaging protocol for an image acquisition, or 3) changes in conditions of the image acquisition comprises:
receiving the input imaging protocol and the one or more performance indicators, the input imaging protocol being for a reference medical image acquisition device; and
determining a target imaging protocol using an image acquisition model based on the at least one of the one or more performance indicators, the input imaging protocol, or the changes in the conditions of the image acquisition comprises:
determining the target imaging protocol using a machine learning based image acquisition model, the machine learning based image acquisition model receives as input the input imaging protocol and the one or more performance indicators and generating as output the target imaging protocol, the target imaging protocol being for a target medical image acquisition device.

18. The non-transitory computer-readable storage medium of claim 15, wherein:
receiving at least one of 1) one or more performance indicators, 2) an input imaging protocol for an image acquisition, or 3) changes in conditions of the image acquisition comprises:
receiving the input imaging protocol and the changes in the conditions of the image acquisition, the input imaging protocol being for an initial condition of the image acquisition; and
determining a target imaging protocol using an image acquisition model based on the at least one of the one or more performance indicators, the input imaging protocol, or the changes in the conditions of the image acquisition comprises:
determining the target imaging protocol using a computational image acquisition model based on the input imaging protocol and the changes in the conditions of the image acquisition, the target imaging protocol being for a target condition of the image acquisition.

19. The non-transitory computer-readable storage medium of claim 15, wherein:
receiving at least one of 1) one or more performance indicators, 2) an input imaging protocol for an image acquisition, or 3) changes in conditions of the image acquisition comprises:
receiving the input imaging protocol and the changes in the conditions of the image acquisition; and
determining a target imaging protocol using an image acquisition model based on the at least one of the one or more performance indicators, the input imaging protocol, or the changes in the conditions of the image acquisition comprises:
determining events using a computational image acquisition model based on the input imaging protocol and the changes in the conditions of the image acquisition.

20. The non-transitory computer-readable storage medium of claim 19, wherein determining a target imaging protocol using an image acquisition model based on the at least one of the one or more performance indicators, the input imaging protocol, or the changes in the conditions of the image acquisition further comprises:
determining the target imaging protocol based on the events.
